(19) United States
(12) Patent Application Publication
(10) Pub. No.: US 2016/0287651 A1
(43) Pub. Date: Oct. 6, 2016

(54) SOLID PREPARATIONS CONTAINING PELARGONIUM SIDOIDES EXTRACTS AND SILICIC ACID COMPOUND, AND PREPARING METHOD THEREOF

(71) Applicant: KOREA UNITED PHARM. INC., Sejong-si (KR)

(72) Inventors: Youn Woong Choi, Gyeonggi-do (KR); Byung Gu Min, Seoul (KR); Sang Min Cho, Gyeonggi-do (KR); Do Hyoung Ki, Gyeonggi-do (KR); Ji Hyun Ahn, Gyeonggi-do (KR); Byung Hoon Lee, Gyeongsangnam-do (KR); Hyung Joon Jun, Seoul (KR); Won Tae Jung, Seoul (KR); Kyu Yeol Nam, Gyeonggi-do (KR); Dong Gyu Lee, Seoul (KR); Jin Seong Chung, Seoul (KR)

(73) Assignee: Korea United Pharm. Inc., Sejong-si (KR)

(21) Appl. No.: 14/889,619

(22) PCT Filed: Dec. 19, 2014
(86) PCT No.: PCT/KR2014/012592

§ 371 Date: Nov. 6, 2015

(30) Foreign Application Priority Data
Dec. 20, 2013 (KR) 10-2013-0159980

(51) Int. Cl. A61K 36/185; A61K 9/20

(52) U.S. Cl. CPC A61K 36/185; A61K 9/2009; A61K 2236/333

(57) ABSTRACT

The present invention relates to a solid preparation including a Pelargonium sidoides extract and a silicic acid compound, which is allowed to be formulated in a solid form by direct adsorption of the Pelargonium sidoides extract onto a silicic acid compound, and a preparation method thereof. Since the solid preparation including the Pelargonium sidoides extract and the silicic acid compound of the present invention has higher stability than a liquid preparation such as syrup, and has no additives such as sugars, there is no concern about microbial contamination or spoilage of the preparation. In addition, it is possible to pack the solid preparation individually. Since the solid preparation is smaller in volume than the liquid preparation, it is highly portable, and there is also a convenience that no additional tools are needed to take the drug. Further, the active ingredient can be taken at the equal amount every time.

### TECHNICAL FIELD

The present invention relates to a solid preparation including a Pelargonium sidoides extract and a silicic acid compound, which is allowed to be formulated in a solid form by direct adsorption of the Pelargonium sidoides extract onto a silicic acid compound, and a preparation method thereof.

### BACKGROUND ART

As recent industrial development accelerates westernization of diets and increases environmental pollution, the prevalence of respiratory diseases such as asthma, bronchitis, allergic rhinitis, etc. is growing. The cause of respiratory disease varies depending on the type, but it is mainly caused by viruses or bacteria. That is, when viruses or bacteria enter the human body via a respiratory tract, substances secreted thereby destroy mucous cells, and viruses or bacteria penetrate into the destroyed cells to cause inflammation, leading to respiratory disease.

To treat respiratory diseases, antibiotics, nasal decongestants, non-steroidal anti-inflammatory agents, antitussive expectorants, etc. are used. Of them, antibiotics have problems that they have side-effects such as loss of appetite, vomit, allergy, etc., and repeated use thereof causes resistance In particular, respiratory disease is more serious in infants or younger children, because it is more common in them than adults, and they are also more sensitive to the side-effects caused by use of antibiotics.

Therefore, there have been many attempts to develop a substance which has a therapeutic effect on respiratory diseases while having fewer side-effects than chemical compounds by extracting it from a natural source. One of them is the use of Pelargonium sidoides.

Pelargonium sidoides grows wild at a high elevation of 2300 m in the inland and coastal regions of South Africa, and has been widely used for treating diarrhea, gastrointestinal diseases, liver diseases, and respiratory diseases such as cold, tuberculosis, etc. for a long time. In particular, it is known that Pelargonium sidoides prevents viruses or bacteria from adhering to mucous cells and the spread of inflammation, thereby showing excellent therapeutic efficacy on respiratory diseases.

Accordingly, therapeutic drugs for respiratory diseases prepared by using a Pelargonium sidoides extract have been developed and marketed. For example, it is sold under the brand name of Kaloba in the UK, Umckan syrup in Brazil, and Umckamin liquid, Umckamin syrup, Umkaroba syrup, or Kukuratum syrup in Korea.

However, since these drugs are prepared and sold in a liquid form, such as syrup, etc., a glycerin mixture should be added, together with the Pelargonium sidoides extract. This glycerin mixture reduces absorption rate of the active ingredient, and thus there is a problem that a large amount of Pelargonium sidoides extract should be used, compared to a solid preparation. In addition, these drugs have a shorter expiry date than a solid preparation, and have a stability problem such as precipitation. Also, microbial contamination and spoilage may occur after opening, because of addition of sugars for sweet taste. Because the liquid preparation should be packed in a separate container for sale, it is less portable than a solid preparation. Further, there is an inconvenience that additional tools such as spoon, cup, etc., are needed to take the liquid drug, and there are also disadvantages that it is difficult to take the equal amount thereof every time, and the container is fragile when dropped.

With regard to a formulation containing the Pelargonium sidoides extract or a preparation method thereof, Korean Patent Publication No. 10-2013-0099549 discloses a pharmaceutical composition containing the Pelargonium extract and sorbic acid or a salt thereof, in which alcohol content is reduced to prevent crystal formation, and Korean Patent Publication No. 10-1140203 discloses a method of preparing a Pelargonium sidoides dry extract using a carrier such as cyclodextrin, maltose, sucrose, etc. However, no solid preparation containing the Pelargonium sidoides extract and no preparation method thereof have been disclosed yet.

### DISCLOSURE OF INVENTION

### Technical Problem

The present inventors have made extensive studies to develop a solid preparation including a Pelargonium sidoides extract. As a result, they found that when the Pelargonium sidoides extract is mixed with a silicic acid compound, the silicic acid compound adsorbs the Pelargonium sidoides extract to prepare a solid preparation, thereby completing the present invention.

An object of the present invention is to provide a solid preparation including a Pelargonium sidoides extract and a silicic acid compound, which has an efficacy equivalent to that of a liquid preparation such as syrup, etc., and shows higher stability and convenient administration than the liquid preparation.

Another object of the present invention is to provide a method for preparing the solid preparation by mixing the Pelargonium sidoides extract with the silicic acid compound.

### Solution to Problem

In one aspect, the present invention provides a solid preparation including a Pelargonium sidoides extract and a silicic acid compound.

In the present invention, Pelargonium sidoides is a perennial plant belonging to Pelargonium sp., and grows wild at a high elevation of 2300 m in the inland and coastal regions of South Africa, and is also called kaloba, umcka or zucol. Pelargonium sidoides has been widely used for diarrhea, gastrointestinal diseases, liver diseases, respiratory diseases such as cold, tuberculosis, etc. for a long time, and in particular, it prevents viruses or bacteria from adhering to mucous cells and the spread of inflammation, thereby showing excellent therapeutic efficacy on respiratory diseases. The Pelargonium sidoides may be purchased from commercially available sources, or collected or grown in the nature, and its flower, seed, stem, root and the whole plant may be used as a raw material.

In the present invention, the Pelargonium sidoides extract refers to a product resulting from extraction of Pelargonium sidoides with a solvent, and includes all of a liquid extract, a fraction of the liquid extract, a crude purified product or a purified product thereof.

The purified product is obtained by removing floating solid particles from the liquid extract or the fraction thereof. The particles are filtered out using cotton, nylon, etc., or ultrafiltration, freezing filtration, centrifugation, or the like may be used, but is not limited thereto. In addition, a separation step by various chromatographies (chromatography based on size, charge, hydrophobicity or affinity) may be further included.

The liquid extract, the fraction thereof, the crude purified product or the purified product thereof may be used in the liquid form as it is, or concentrated and/or dried before use. The concentrating and/or drying method includes, but is not limited to, freeze drying, vacuum drying, hot air drying, spray drying, drying under reduced pressure, foam drying, high frequency drying, infrared drying, or the like.

In the present invention, the extraction method of the Pelargonium sidoides extract is not particularly limited, as long as it is a method for extracting an active ingredient from Pelargonium sidoides, and for example, hot water extraction, cold immersion extraction, ultrasonic extraction, reflux cooling extraction or the like may be used.

In the present invention, the solvent for the extraction means C₁-C₄ alcohol such as methanol, ethanol, propanol, or butanol, or an aqueous solution thereof, hexane, ethyl acetate, acetone, methylene chloride dichloromethane, N,N-dimethylformamide (DMF), dimethyl sulfoxide (DMSO), polyol such as 1,3-butylene glycol or propylene glycol, or water singly or in a mixture of two or more thereof, but the type is not limited thereto. Methanol or ethanol is preferred, and ethanol is more preferred.

In one specific embodiment, the dry root of Pelargonium sidoides was washed and cut, and then extracted with ethanol to obtain the Pelargonium sidoides extract.

In the present invention, the silicic acid compound is a compound containing silicic acid therein, and it means calcium silicate, sodium silicate, colloidal silicon dioxide, potassium silicate, magnesium silicate or aluminium magnesium silicate singly or in a mixture of two or more thereof, but the type is not limited thereto. Preferably, the compound includes one or more of calcium silicate, colloidal silicon dioxide and aluminium magnesium silicate, and most preferably, calcium silicate.

The silicic acid compound adsorbs the Pelargonium sidoides extract when they are mixed with each other, which allows preparation of Pelargonium sidoides extract into a solid preparation.

In a specific embodiment, each of calcium silicate, colloidal silicon dioxide, and aluminium magnesium silicate was mixed with the Pelargonium sidoides extract to prepare a solid preparation, respectively. As a result, the solid preparation group prepared by using calcium silicate as an adsorbent showed lower friability and less production of fine powder than other preparation groups, and they were economical while there was almost no variation between the prepared solid preparations.

The Pelargonium sidoides extract and the silicic acid compound are preferably mixed at a weight ratio of 1:0.5 to 1:6 (w/w), and more preferably 1:1.5 to 1:5, based on the dry weight of the Pelargonium sidoides extract. If the weight ratio (w/w) of the Pelargonium sidoides extract and the silicic acid compound is less than 1:0.5, the adsorption of the Pelargonium sidoides extract by the silicic acid compound is weak, and the hardness of the prepared solid preparation is increased to prolong the disintegration time, resulting in delayed onset of efficacy. If the weight ratio (w/w) of the Pelargonium sidoides extract and the silicic acid compound is more than 1:6, there is no difference in adsorption of the Pelargonium sidoides extract, compared to the lower weight ratio, and the amount of silicic acid compound added is excessively large and thus the prepared solid preparation has low hardness and high friability, which is uneconomical. In addition, variation between the prepared solid preparations may occur due to production of fine powder.

In a specific embodiment, calcium silicate was mixed at a weight ratio of 1:0.5, 1.5, 2, 2.5, 3.5, 5 and 6, with respect to the dry weight of the Pelargonium sidoides extract to prepare solid preparations. As a result, when calcium silicate was mixed at a weight ratio of 1:1.5 to 1:5, with respect to the dry weight of the Pelargonium sidoides extract, the prepared solid preparations were economically prepared, because they had proper hardness and low friability. It was also observed that the prepared solid preparations were uniformly coated, and there was almost no variation between the prepared solid preparations.

In the present invention, the solid preparation refers to a formulation having a shape in a solid state, and examples thereof may include a tablet, a pill, a capsule, a powder or a granule, but the type is not limited thereto.

The tablet refers to a product which is prepared by compressing a drug into a particular shape, the pill refers to a product which is obtained by preparing a drug in a spherical shape, and the capsule refers to a product which is prepared by filling a powder- or granule-shaped drug in a capsule or by encapsulating it with capsule base. The powder refers to a mixture of finely divided drugs or chemicals or a composition thereof in a dry form, and the granule refers to a product which is obtained by preparing a medicine or medical mixture in a particle shape.

In the present invention, the solid preparation may be used for the purpose of treating respiratory diseases.

The respiratory disease means a disease caused by inflammation which occurs after viruses or bacteria enter the human body via a respiratory tract. Examples thereof may include acute/chronic infectious disease, bronchitis, sinusitis, tonsillitis, rhinopharyngitis, tympanitis, cough, runny nose, nasal congestion, sore throat, and fever, but the type is not limited thereto.

In another aspect, the present invention provides a method for preparing the solid preparation by mixing the Pelargonium sidoides extract with the silicic acid compound.

In detail, the present invention provides a method for preparing the solid preparation by mixing the Pelargonium sidoides extract with the silicic acid compound, including the steps of:

(a) adding the Pelargonium sidoides extract and the silicic acid compound to a high speed mixer and mixing them with each other to adsorb the Pelargonium sidoides extract onto the silicic acid compound, thereby preparing an adsorption product;

(b) adding an excipient and a binder to the adsorption product of (a) and mixing them with each other to prepare a mixture;

(c) drying and sieving the mixture of (b) to prepare a sieved product;

(d) adding a disintegrant and a lubricant to the sieved product of (c) and mixing them with each other to prepare a mixture;

(e) tabletting the mixture of (d) using a tabletting machine to prepare a tabletted product; and

(f) adding Opadry to the tabletted product of (e) to perform a coating process.

In the present invention, the Pelargonium sidoides extract and the silicic acid compound are the same as described above.

The type of the excipient is not limited, as long as it is pharmaceutically acceptable and functions to increase the volume for making a solid preparation with a desired size, and examples thereof may include lactose, starch, white sugar, mannitol, sorbitol, microcrystalline cellulose or the like. Lactose hydrate, microcrystalline cellulose, or a mixture thereof is preferred.

The binder functions to increase adhesion of the particles to aid granulation and also to maintain physical shape of the final molded product. The type thereof is not limited, as long as it is pharmaceutically acceptable. Examples thereof may include white sugar, glucose, starch, gelatin, Arabia rubber, povidone or the like. Povidone is preferred.

The disintegrant functions to absorb water to facilitate disintegration of the solid preparation into small particles upon intake of the solid preparation. The type thereof is not limited, as long as it is pharmaceutically acceptable. Examples thereof may include crystalline cellulose, starch, croscarmellose sodium or the like. Croscarmellose sodium is preferred.

The lubricant functions to improve fluidity of the sieved product to decrease friction between the sieved product and the tabletting machine, thereby helping compression and release of the prepared solid preparation. The type thereof is not limited, as long as it is pharmaceutically acceptable. Examples thereof may include stearic acid, stearate, talc, carnauba wax, sodium stearyl fumarate, colloidal silicon dioxide, magnesium silicate or the like. Sodium stearyl fumarate, colloidal silicon dioxide, or a mixture thereof is preferred.

In the present invention, the Pelargonium sidoides extract and the silicic acid compound are preferably mixed at a weight ratio (w/w) of 1:0.5 to 1:6, and more preferably, 1:1.5 to 1:5, based on the dry weight of the Pelargonium sidoides extract to prepare the solid preparation.

The solid preparation including the Pelargonium sidoides extract and the silicic acid compound which is prepared by the above method prevents viruses or bacteria from adhering to mucous cells and the spread of inflammation, thereby being used for the treatment of respiratory diseases such as acute/chronic infectious disease, bronchitis, sinusitis, tonsillitis, rhinopharyngitis, tympanitis, cough, runny nose, nasal congestion, sore throat, fever or the like.

### Advantageous Effects of Invention

A solid preparation including a Pelargonium sidoides extract and a silicic acid compound of the present invention has fewer side-effects than chemical compounds because it is extracted from the natural source, Pelargonium sidoides. Accordingly, there is no concern about safety problems and resistance developing, and thus it can be safely used for infants.

Further, since it has higher stability than a liquid preparation such as syrup, and has no additives such as sugars, there is no concern about microbial contamination or spoilage of the preparation. In addition, it is possible to pack the solid preparation individually. Since the solid preparation is smaller in volume than the liquid preparation, it is highly portable, and there is also a convenience that no additional tools such as spoon, cup, etc., are needed to take the drug.

Further, since it is not necessary to add an additive such as glycerin other than the Pelargonium sidoides extract, there are advantages that the preparation process is simple and the active ingredient can be taken at the equal amount every time, unlike the liquid preparation.

### MODE FOR THE INVENTION

Hereinafter, the present invention will be described in more detail with reference to Examples. However, it is apparent to those skilled in the art that these Examples are for illustrative purposes only, and the scope of the present invention is not intended to be limited by these Examples.

### Example 1

### Preparation of Pelargonium sidoides Extract

The dry roots of Pelargonium sidoides were cut into a size of approximately 10 mm or less, and then immersed in 35% ethanol. 5.3% ethanol was added thereto at a volume of approximately 8 times that of the dry root. Thereafter, the liquid extract thus prepared was filtered, and then sterilized at 120 to 121° C. for approximately 30 seconds, and then cooled to prepare a Pelargonium sidoides extract.

### Example 2

### Preparation of Solid Preparation Including Pelargonium sidoides Extract and Silicic Acid Compound

The Pelargonium sidoides extract extracted in Example 1 and a silicic acid compound were put in a high speed mixer and they were mixed with each other to adsorb the Pelargonium sidoides extract onto the silicic acid compound. Thereafter, microcrystalline cellulose and lactose hydrate as excipients and povidone as a binder were added to the adsorption product, and mixed with each other. The mixture was dried and sieved. Next, croscarmellose sodium as a disintegrant and colloidal silicon dioxide and sodium stearyl fumarate as lubricants were added to the sieved product, and mixed with each other. The mixture was tabletted using a tabletting machine. Then, Opadry was added to the tabletted product to perform a coating process. Finally, a solid preparation including the Pelargonium sidoides extract and the silicic acid compound were prepared.

### Example 3

### Comparison of Properties Between Solid Preparations Including Pelargonium sidoides Extract and Silicic Acid Compound According to Type of Silicic Acid Compound

3-1. Preparation of Solid Preparation Using Silicic Acid Compound as Adsorbent

To compare properties between the solid preparations which were prepared by using the Pelargonium sidoides extract and different types of silicic acid compounds, 20 mg of the Pelargonium sidoides extract extracted in Example 1 and each 50 mg of calcium silicate, colloidal silicon dioxide, or aluminium magnesium silicate were mixed, respectively. At this time, the content of the Pelargonium sidoides extract was expressed based on the dry weight of the extract. Thereafter, solid preparations were prepared using the components as in the content of the following Table 1, before the coating step in the method of Example 2.

TABLE 1

| Ingredient (mg) | Use | A | B | C |
|---|---|---|---|---|
| Pelargonium sidoides extract | Main ingredient | 20 | 20 | 20 |
| Calcium silicate | Adsorbent | 50 | — | — |
| Colloidal silicon dioxide | | — | 50 | — |
| Aluminium magnesium silicate | | — | — | 50 |
| Microcrystalline cellulose | Excipient | 185 | 185 | 185 |
| Lactose hydrate | | 110 | 110 | 110 |
| Povidone | Binder | 10 | 10 | 10 |
| Croscarmellose sodium | Disintegrant | 10 | 10 | 10 |
| Colloidal silicon dioxide | Lubricant | 10 | 10 | 10 |
| Sodium stearyl fumarate | | 15 | 15 | 15 |
| Total weight | | 410 | 410 | 410 |

3-2. Comparison of Properties Between Solid Preparations Prepared by Using Different Types of Silicic Acid Compounds

To compare properties between solid preparations prepared by using the Pelargonium sidoides extract and different types of silicic acid compounds, thickness, hardness, friability and disintegration time of the solid preparations prepared in Example 3-1 were measured, and the results are shown in the following Table 2.

TABLE 2

| | A | B | C |
|---|---|---|---|
| Thickness (mm) | 5.8 | 4.5 | 6.0 |
| Hardness (kPa) | 12.0 | 5 | 6 |
| Friability (%) | 0.17 | 1.2 | 1.0 |
| Disintegration time (min) | 10 | 6 | 7 |

The experimental results showed that a solid preparation prepared by using colloidal silicon dioxide as the adsorbent had the lowest thickness and hardness of all other groups, and thus the sieved products were sticky, and sticking occurred during tabletting, resulting in an irregular solid preparation. In addition, its friability was 1.2%, which was approximately 7.1 times higher than that of a solid preparation prepared by using calcium silicate, and therefore, there was a lot of loss during preparation of the solid preparation, and dust particles were produced during the preparation process, leading to formation of irregular surface by uneven coating result. In addition, a large amount of fine powder was generated to deteriorate fluidity upon tabletting, leading to a weight-difference in the solid preparations.

Further, the solid preparation prepared by using aluminium magnesium silicate as the adsorbent had the greatest thickness of 6 mm, and its hardness was 6 kPa which was slightly higher than that of the solid preparation prepared by using colloidal silicon dioxide, but it was only half the hardness of the group prepared by using calcium silicate. Its friability was 1%, and the loss rate was lower than that of the group prepared by using colloidal silicon dioxide, but approximately 5.9 times higher than that of the group prepared by using calcium silicate, which indicates that there was much loss during preparation of the solid preparation.

In contrast, a solid preparation prepared by using calcium silicate as the adsorbent had the thickness of 5.8 mm, which was slightly thinner than the group prepared by using aluminium magnesium silicate. Its hardness was 12 kPa, which was the greatest hardness among the experimental groups, and sticking hardly occurred due to the strongest adhesion force between sieved products. Therefore, there was almost no variation between the prepared solid preparations. In addition, its friability was 0.17%, which was the lowest friability among the experimental groups. The loss rate was low and little fine powder was produced, leading to a uniform coating result.

It was found that although the group prepared by using calcium silicate as the adsorbent had higher hardness than the group prepared by using aluminium magnesium silicate or colloidal silicon dioxide, its disintegration time was not delayed.

### Example 4

### Comparison of Properties Between Solid Preparations Including Pelargonium sidoides Extract and Calcium Silicate According to Amount of Calcium Silicate

4-1. Preparation of Solid Preparation Using Calcium Silicate as the Adsorbent

To compare properties between the solid preparations which were prepared by using the Pelargonium sidoides extract and different amounts of calcium silicate, 20 mg of the Pelargonium sidoides extract extracted in Example 1 and 10, 30, 40, 50, 70, 100 or 120 mg of calcium silicate were mixed, respectively. At this time, the content of the Pelargonium sidoides extract was expressed based on the dry weight of the extract. Thereafter, solid preparations were prepared using the components as in the content of the following Table 3, before the coating step in the method of Example 2.

TABLE 3

| Ingredient (mg) | A | B | C | D | E | F | G |
|---|---|---|---|---|---|---|---|
| Pelargonium sidoides | 20 | 20 | 20 | 20 | 20 | 20 | 20 |
| extract | | | | | | | |
| Calcium silicate | 10 | 30 | 40 | 50 | 70 | 100 | 120 |
| Microcrystalline | 185 | 185 | 185 | 185 | 185 | 185 | 185 |
| cellulose | | | | | | | |
| Lactose hydrate | 110 | 110 | 110 | 110 | 110 | 110 | 110 |
| Povidone | 10 | 10 | 10 | 10 | 10 | 10 | 10 |
| Croscarmellose | 10 | 10 | 10 | 10 | 10 | 10 | 10 |
| sodium | | | | | | | |
| Colloidal silicon | 10 | 10 | 10 | 10 | 10 | 10 | 10 |
| dioxide | | | | | | | |
| Sodium stearyl | 15 | 15 | 15 | 15 | 15 | 15 | 15 |
| fumarate | | | | | | | |
| Total weight | 370 | 390 | 400 | 410 | 430 | 460 | 480 |

4-2. Comparison of Properties Between Solid Preparations Prepared by Using Different Amounts of Calcium Silicate

To compare properties between solid preparations prepared by using the Pelargonium sidoides extract and different amounts of calcium silicate, thickness, hardness, friability and disintegration time of the solid preparations prepared in Example 4-1 were measured, and the results are shown in the following Table 4.

TABLE 4

| | A | B | C | D | E | F | G |
|---|---|---|---|---|---|---|---|
| Thickness (mm) | 5.5 | 5.6 | 5.7 | 5.8 | 6.0 | 6.3 | 6.5 |
| Hardness (kPa) | — | 14 | 12.5 | 12.0 | 11.6 | 7 | 3 |
| Friability (%) | 0.05 | 0.1 | 0.2 | 0.17 | 0.2 | 0.5 | 1.8 |
| Disintegration time (min) | 30 | 15 | 10 | 10 | 10 | 8 | 5 |

The experimental results showed that as the amount of calcium silicate was increased, hardness of the prepared solid preparations was decreased. In a solid preparation prepared by mixing 10 mg of calcium silicate, the sieved products became sticky during preparation of the solid preparation, and thus it is hard to measure its hardness. In a solid preparation prepared by mixing 120 mg thereof, its hardness was as very low as 3, being crumbly.

With regard to friability, as the amount of calcium silicate was increased, friability of the prepared solid preparations was increased. In a solid preparation prepared by mixing 100 mg or less of calcium silicate, its friability was 0.5% or less, indicating low loss rate during preparation of the solid preparation. Little fine powder was produced, leading to a uniform coating result of the solid preparation. However, in the group prepared by mixing 120 mg of calcium silicate, the prepared sieved products became dry and a large amount of fine powder was produced and thus capping occurred during tabletting. In addition, its friability was 1.8%, which was approximately 3.6 times higher than the group prepared by mixing 100 mg thereof.

The disintegration time tended to gradually decrease, as the amount of calcium silicate was increased. In particular, the solid preparation prepared by mixing 10 mg of calcium silicate showed the disintegration time of 30 minutes, indicating delayed disintegration compared to other groups.

1. A solid preparation comprising a Pelargonium sidoides extract and a silicic acid compound.

2. The solid preparation according to claim 1, wherein the silicic acid compound is one or more selected from the group consisting of calcium silicate, colloidal silicon dioxide and aluminium magnesium silicate.

3. The solid preparation according to claim 1, wherein the silicic acid compound adsorbs the Pelargonium sidoides extract.

4. The solid preparation according to claim 1, wherein the extract is obtained by performing extraction using one selected from the group consisting of water, ethanol, methanol, propanol and butanol, or a mixture of two or more thereof as a solvent.

5. The solid preparation according to claim 1, wherein the Pelargonium sidoides extract and the silicic acid compound are mixed at a weight ratio of 1:0.5 to 1:6.

6. The solid preparation according to claim 1, wherein the solid preparation is one or more selected from the group consisting of a tablet, a pill, a capsule, a powder and a granule.

7. The solid preparation according to claim 1, wherein the solid preparation is used for the treatment of respiratory diseases.

8. The solid preparation according to claim 7, wherein the respiratory disease is one or more selected from the group consisting of cold, cough, asthma, tonsillitis, sore throat, tuberculosis, and chronic bronchitis.

9. A method for preparing the solid preparation according to claim 1 by mixing a Pelargonium sidoides extract with a silicic acid compound.

10. The method according to claim 9, wherein the Pelargonium sidoides extract and the silicic acid compound are mixed at a weight ratio of 1:0.5 to 1:6.

11. A method for treating a respiratory disease, comprising administering the solid preparation of claim 1 comprising a Pelargonium sidoides extract and a silicic acid compound to a subject in need thereof.

12. The method of claim 11, wherein the respiratory disease is one or more selected from the group consisting of cold, cough, asthma, tonsillitis, sore throat, tuberculosis, and chronic bronchitis.

13. The method of claim 11, wherein the silicic acid compound is one or more selected from the group consisting of calcium silicate, colloidal silicon dioxide and aluminium magnesium silicate.

14. The method of claim 11, wherein the silicic acid compound adsorbs the Pelargonium sidoides extract.

15. The method of claim 11, wherein the extract is obtained by performing extraction using one selected from the group consisting of water, ethanol, methanol, propanol and butanol, or a mixture of two or more thereof as a solvent.

16. The method of claim 11, wherein the Pelargonium sidoides extract and the silicic acid compound are mixed at a weight ratio of 1:0.5 to 1:6.

17. The method of claim 11, wherein the solid preparation is one or more selected from the group consisting of a tablet, a pill, a capsule, a powder and a granule.

18. The method of claim 11, wherein the solid preparation is a tablet comprising a Pelargonium sidoides extract and calcium silicate, at a weight ratio of 1:1.5 to 1:5.

19. A tablet comprising a Pelargonium sidoides extract and calcium silicate, at a weight ratio of 1:1.5 to 1:5.

20. A method for preparing a tablet by mixing a Pelargonium sidoides extract with calcium silicate, at a weight ratio of 1:1.5 to 1:5.
